(19) United States
(12) Patent Application Publication
(10) Pub. No.: US 2026/0182540 A1
(43) Pub. Date: Jul. 2, 2026

(54) ANIMAL TRACKING BASED ON CHEMICAL SIGNATURE

(71) Applicant: International Business Machines Corporation, Armonk, NY (US)

(72) Inventor: Sanmitra Sarkar, Kolkata (IN)

(21) Appl. No.: 19/007,733

(22) Filed: Jan. 2, 2025

(51) Int. Cl. A01K 11/00; G16C 20/70

(52) U.S. Cl. CPC A01K 11/008; G16C 20/70

(57) ABSTRACT

According to an embodiment of the present invention, a computer system comprises a processor set, one or more computer-readable storage media, and program instructions stored on the one or more computer-readable storage media to cause the processor set to perform operations. The system receives information from sensors at different locations indicating detection of chemicals from a plurality of animals in proximity of the sensors. A machine learning model determines presence of a pattern of chemicals associated with an animal within the information from the sensors. One or more locations of the animal are identified based on the sensors detecting the pattern of chemicals associated with the animal. Embodiments of the present invention further include a method and computer program product for animal tracking in substantially the same manner described above.

### BACKGROUND

### 1. Technical Field

Present invention embodiments relate to location tracking, and more specifically, to tracking locations of an animal based on a chemical signature to search for a lost animal.

### 2. Discussion of the Related Art

People become attached to their pets and often consider them members of their family. However, while a family is traveling, or simply loses sight of their pet for a moment, the pet can go missing. In some cases, the pet cannot trace their way back to their owner's home. Further, the pet may be subject to a crime that causes the pet to be missing. In these instances, determining past and present locations of the pet can be difficult.

### SUMMARY

According to an embodiment of the present invention, a computer system comprises a processor set, one or more computer-readable storage media, and program instructions stored on the one or more computer-readable storage media to cause the processor set to perform operations. The system receives information from sensors at different locations indicating detection of chemicals from a plurality of animals in proximity of the sensors. A machine learning model determines presence of a pattern of chemicals associated with an animal within the information from the sensors. One or more locations of the animal are identified based on the sensors detecting the pattern of chemicals associated with the animal. Embodiments of the present invention further include a method and computer program product for animal tracking in substantially the same manner described above.

### BRIEF DESCRIPTION OF THE DRAWINGS

Generally, like reference numerals in the various figures are utilized to designate like components.

FIG. 1 is a diagrammatic illustration of an example computing environment according to an embodiment of the present invention.

FIG. 2 is a procedural flowchart of a method of animal tracking according to an embodiment of the present invention.

FIG. 3 is an example graph of a Fourier transformation of digitized olfaction data according to an embodiment of the present invention.

FIG. 4 is an example time series based cyclic graph of an animal inside a bounded region according to an embodiment of the present invention.

FIG. 5 is a flow diagram of a manner of animal tracking according to an embodiment of the present invention.

### DETAILED DESCRIPTION

An embodiment of the present invention finds any lost pet or animal (e.g., dog, cat, bird, rabbit, horse/pony, household pet, zoo or wild animal, circus animal, farm animal, etc.) using a multisensory system and an olfactory signature of an animal. A lost pet or animal is found without the aid of any trained animals by digitizing olfactory signals of animals detected by sensors and maintaining a database to enable traceability of a lost pet or animal. By way of example, the olfactory sensors may be disposed in a smart city or other bounded region.

An embodiment of the present invention identifies whether a particular pet or animal was present at a specific time and position, given a combination of chemical signals being emanated from several animals over a given period of time, and upon analysis of a digitized spectrum of olfactory signals exhibited by those chemicals.

An embodiment of the present invention collects an olfactory signature by digitizing the olfactory signature as a spectrum of multiple olfactory signatures. The digitized signature is a pattern which is specific and a mix, in both time and frequency domain, of known olfactory signatures.

An embodiment of the present invention determines the olfactory signature of a lost pet or animal from its clothes, garments, collars, leashes, and/or ornaments associated with and used by the pet or animal. The pet or animal's presence is detected in a non-intrusive way, without visual, auditory, fingerprinting or other intrusive activities, simply by analyzing the olfactory signature.

An embodiment of the present invention creates a directed cyclic graph of locations of the lost pet or animal from the detected presence based on analysis of the olfactory signature of the lost pet or animal within a bounded region. A plurality of directed cyclic graphs may be generated for different bounded regions. The traversal path of the lost pet or animal may be traced from the directed cyclic graph of the lost pet or animal by joining the nodes and providing any missing directional pointers for the locations. Further, the tracing may be performed in combination with other devices (e.g., CCTV, etc.).

An embodiment of the present invention may ascertain additional information about a pet or animal at the time of identification besides the animal presence, such as information about diseases, behavior, emotional state, and/or health status (e.g., sick, excited, sleeping, etc.).

An embodiment of the present invention tracks a pet or animal in real time by synchronizing an identification of the lost pet or animal based on a digitized olfactory signature and a Global Navigation Satellite System (GNSS). Integration of olfactory sensors with GNSS is activated and paired with required satellites during initiation. The olfactory sensors transmit and receive geo position information with the satellites once the target olfactory signature is detected.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer-readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer-readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

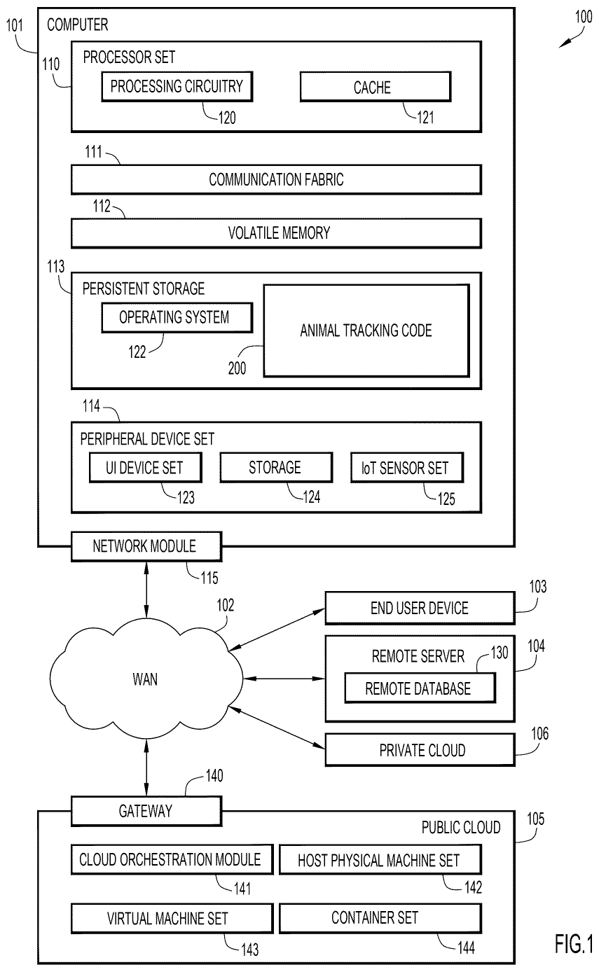

Referring to FIG. 1, computing environment 100 contains an example of an environment for the execution of at least some of the computer code involved in performing the inventive methods, such as animal tracking code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer-readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer-readable program instructions are stored in various types of computer-readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer-readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

CLOUD COMPUTING SERVICES AND/OR MICROSERVICES (not separately shown in FIG. 1): public and private clouds 105, 106 are programmed and configured to deliver cloud computing services and/or microservices (unless otherwise indicated, the word “microservices” shall be interpreted as inclusive of larger “services” regardless of size). Cloud services are infrastructure, platforms, or software that are typically hosted by third-party providers and made available to users through the internet. Cloud services facilitate the flow of user data from front-end clients (for example, user-side servers, tablets, desktops, laptops), through the internet, to the provider's systems, and back. In some embodiments, cloud services may be configured and orchestrated according to an “as a service” technology paradigm where something is being presented to an internal or external customer in the form of a cloud computing service. As-a-Service offerings typically provide endpoints with which various customers interface. These endpoints are typically based on a set of APIs. One category of as-a-service offering is Platform as a Service (PaaS), where a service provider provisions, instantiates, runs, and manages a modular bundle of code that customers can use to instantiate a computing platform and one or more applications, without the complexity of building and maintaining the infrastructure typically associated with these things. Another category is Software as a Service (SaaS) where software is centrally hosted and allocated on a subscription basis. SaaS is also known as on-demand software, web-based software, or web-hosted software. Four technological sub-fields involved in cloud services are: deployment, integration, on demand, and virtual private networks.

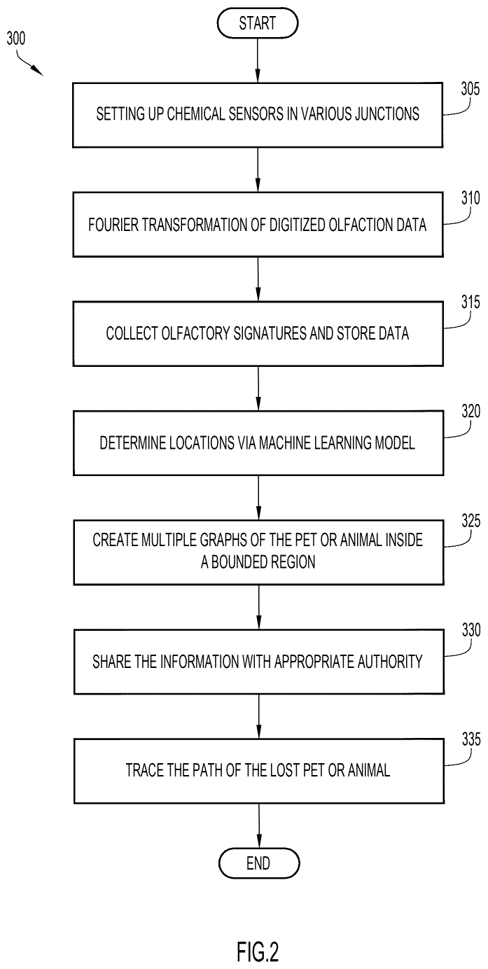

A method 300 of animal tracking (e.g., via animal tracking code 200, computer 101, etc.) according to an embodiment of the present invention is illustrated in FIG. 2. Initially, chemical or olfactory sensors (e.g., olfactometer, E-noses, etc.) are disposed at various junctions or locations at operation 305. By way of example, the chemical sensors may be any conventional or other sensing devices (e.g., olfactometer, E-noses, etc.), and can be disposed at various crossings, roads, buildings, highways, etc. The chemical sensors detect chemicals from various pets or animals passing near the sensors at various points of time from various junctions. The chemical sensors measure the presence and intensity of the different detected chemicals.

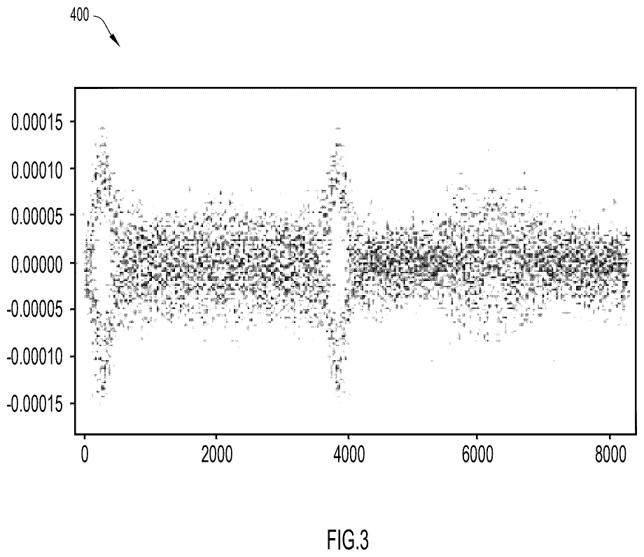

The chemical sensors provide digitized data, and a Fourier transformation is performed on the digitized data at operation 310. The Fourier transformation may be performed using any conventional or other Fourier transformation (e.g., FFT, etc.). The Fourier transformation analyzes a time-based pattern (e.g., digitized sensor data, etc.), measures every possible detected smell (or chemical), and returns an overall pattern of the smells or chemicals (or olfactory pattern or signature) (FIG. 3). The olfactory pattern varies when different smells, pheromones, etc. pass by the sensor location.

The chemical sensors include one or more sensors(e.g., E-noses, etc.) that smell different smells, pheromones, etc. in a locality and one or more sensors (e.g., olfactometer, etc.) that can measure the presence or absence of certain chemicals. The chemical sensors may also measure the power and intensity of the smell and a provide a timestamp indicating a time of detection. The olfactory patterns or signatures are collected through the chemical sensors (e.g., olfactometer, E-noses, etc.) and stored (e.g., in a database, cloud, etc.) at operation 315. Information is extracted from the olfactory data transformed through the Fourier transformation to provide an indication of the chemicals and associated information (e.g., presence, intensity, presence of an animal, etc.). For example, the chemicals from an olfactory pattern may be provided in a table with each row corresponding to a detection with columns corresponding to chemicals and a presence indication of an animal (e.g., a Methyl Hexenoic Acid (MHA) column indicating a presence/absence of the chemical (e.g., Yes or No, etc.), an Isovaleric Acid (IA) column indicating a presence/absence of the chemical (e.g., Yes or No, etc.), a Methanethiol (MT) column indicating a presence/absence of the chemical (e.g., Yes or No, etc.), an MHA Intensity (OU/m3) column indicating a numeric measure of the intensity or concentration of the chemical, an IA Intensity(OU/m3) column indicating a numeric measure of the intensity or concentration of the chemical, an MT Intensity (OU/m3) indicating a numeric measure of the intensity or concentration of the chemical, and a presence column indicating presence/absence of an animal based on the measured chemicals (e.g., Yes or No, etc.). However, any chemicals and measurements may be provided in the table. By way of example, an animal may be indicated as present when MHA is present with an intensity of 10 and IA is present with an intensity of 5, while an animal may be indicated as absent when MHA is present with an intensity of 2 and IA is present with an intensity of 1. The table and other data for a stream (e.g., chemicals, intensities, timestamps, sensor or location identifiers, etc.) are stored (e.g., in a database, uploaded to the cloud, etc.).

One or more locations of a lost pet or animal are determined via a machine learning model at operation 320. The machine learning model receives an olfactory signature of a lost pet or animal and identifies the presence of the olfactory signature of the lost pet or animal in the data from the sensors. The data may include a sensor or location identifier to indicate the origin of the data. The olfactory signature of the lost pet or animal may be obtained from its clothes, garments, belts, ornaments, etc. (e.g., which may be subject to permission of its owner) in substantially the same manner described above. The location of the sensor detecting the signature indicates the location of the lost pet or animal while the corresponding timestamp indicates a time of the lost pet or animal at that location. The collected data from each sensor may be applied individually (e.g., sensor data from a first location, sensor data from a second location, etc.) to determine the sensor (and location) detecting the olfactory signature of the lost pet or animal.

The machine learning model may include any conventional or other machine learning models (e.g., mathematical/statistical, classifiers, feed-forward, recurrent, convolutional, deep learning, or other neural networks, large language models (LLM), etc.). By way of example, the machine learning model may include one or more artificial neural networks. For example, neural networks may include an input layer, one or more intermediate layers (e.g., including any hidden layers), and an output layer. Each layer includes one or more neurons, where the input layer neurons receive input (e.g., data or features, etc.), and may be associated with weight values. The neurons of the intermediate and output layers are connected to one or more neurons of a preceding layer, and receive as input the output of a connected neuron of the preceding layer. Each connection is associated with a weight value, and each neuron produces an output based on a weighted combination of the inputs to that neuron. The output of a neuron may further be based on a bias value for certain types of neural networks (e.g., recurrent types of neural networks).

The weight (and bias) values may be adjusted based on various training techniques. For example, the machine learning of the neural network may be performed using a training set of various example data, features, and/or information as input (e.g., olfactory signature, collection of olfactory signatures, features of the olfactory signatures, etc.) and corresponding desired outputs (e.g., presence or absence of the input olfactory signature in the collection of olfactory signatures), where the neural network attempts to produce the provided output and uses an error from the output (e.g., difference between produced and known outputs) to adjust weight (and bias) values (e.g., via backpropagation or other training techniques).

The output layer neurons may indicate a probability for the input data being associated with a corresponding output (e.g., presence or absence of an olfactory signature in a collection of olfactory signatures). The output with the highest probability may be selected as the result.

The artificial neural network may use a multilayer perceptron to classify whether the lost pet or animal has actually passed the locality recently. A set of input variables for the artificial neural network of presence and absence of certain chemicals, intensity of chemicals, etc. can be provided to the neural network based on the olfactory signature of the lost pet or animal and the table data. From a given collection of raw data points, given a certain pattern to look for (depending upon factors like the pet or animal's known olfactory signature), the system determines with a certain confidence (probability) a yes/no answer to the question of whether that pet or animal had passed through that point or location.

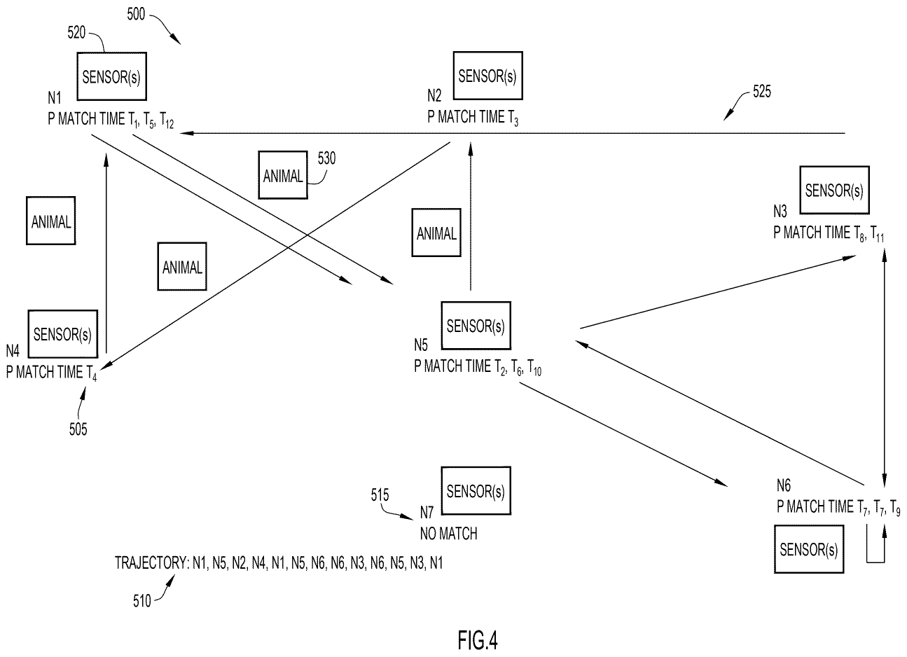

Time series based cyclic graphs of the lost pet or animal inside a bounded region is created at operation 325. These graphs are applied for different bounded regions to create multiple graphs. The graphs (FIG. 4) include nodes representing sensors at locations for detecting the lost pet or animal, and edges or pointers indicating paths traversed by the lost pet or animal. The paths are preferably indicated in chronological order (based on the timestamps).

The location information may be shared with appropriate authorities (e.g., police, etc.) at operation 330. The information may include the most recent location of the lost pet or animal, the history of the places the pet or animal traversed, and/or the global map of the pet or animal trajectory.

The path of the lost pet or animal (or olfactory signature) can be traced at operation 335. For example, the various location points of the chemical sensors that have already confirmed the presence of the olfactory signature may be joined by lines to determine the paths that the lost pet or animal traversed. Other devices (e.g., CCTV, etc.) may be used to confirm the identity and trace the lost pet or animal. By way of example, closed-circuit television (CCTV) may provide images of the animal detected at various locations to confirm the animal is the lost pet or animal (e.g., owner verify, compare an image of the lost animal to CCTV images manually or via conventional or other image processing techniques, etc.). Further, the locations and timestamps may be used as indexes to search through the voluminous data produced by the CCTV to quickly identify the appropriate footage data and improve computer performance.

Moreover, an embodiment of the present invention may ascertain additional information about a pet or animal at the time of identification besides the animal presence, such as information about diseases, behavior, emotional state, and health status (e.g., sick, excited, sleeping, etc.). This may be determined at the time of detection based on the information from the sensors corresponding to the olfactory signature of the animal. For example, the presence of certain chemicals and their intensity may be mapped or associated with certain conditions of the animal (e.g., sick, excited, sleeping, etc.).

In addition, a pet or animal may be tracked in real time by synchronizing an identification of the lost pet or animal and a Global Navigation Satellite System (GNSS). Integration of olfactory sensors with GNSS is activated and paired with required satellites during initiation. The olfactory sensors transmit and receive geo position with the satellites once the target olfactory signature is detected. This enables more accurate and precise tracking of the lost pet or animal.

An example graph 400 of a Fourier transformation of digitized olfaction data according to an embodiment of the present invention is illustrated in FIG. 3. The Fourier transformation basically converts the signals from the chemical sensors in the time domain to the frequency domain. The time domain signal is composed of multiple signals representing the different chemicals detected. The Fourier transformation converts the time domain signal to the frequency domain to indicate the frequency components of the time domain signal (representing the chemicals). The time domain signal may be of any time interval (e.g., milliseconds, seconds, etc.) for the Fourier transformation.

Graph 400 includes an X-axis corresponding to frequencies (or chemicals) and a Y-axis showing an amplitude (or intensity). The combination of the chemicals and intensities forms the olfactory pattern or signature. Thus, the conversion shows the chemicals in the signal corresponding to the frequencies.

An example time series based cyclic graph 500 of an animal 530 inside a bounded region according to an embodiment of the present invention is illustrated in FIG. 4. The time series based graph tracks the movement of animal 530 over a specified time interval or period based on sensors 520 detecting the presence of the lost pet or animal. Graph 500 includes sensors or nodes 520 (e.g., N as viewed in FIG. 4) at various locations for detecting lost pet or animal 530, and edges or pointers 525 indicating paths (and direction) traversed by the lost pet or animal between the nodes. The nodes are preferably associated with times of detection of the lost pet or animal represented by a variable 505 (e.g., T as viewed in FIG. 4), and an indication of a presence of the lost pet or animal represented by a variable 515 (e.g., P as viewed in FIG. 4). A trajectory 510 may be provided indicating the path traversed by the lost pet or animal. The trajectory includes a listing of nodes 520 (e.g., N as viewed in FIG. 4) detecting the lost pet or animal, preferably in chronological order based on the timestamps.

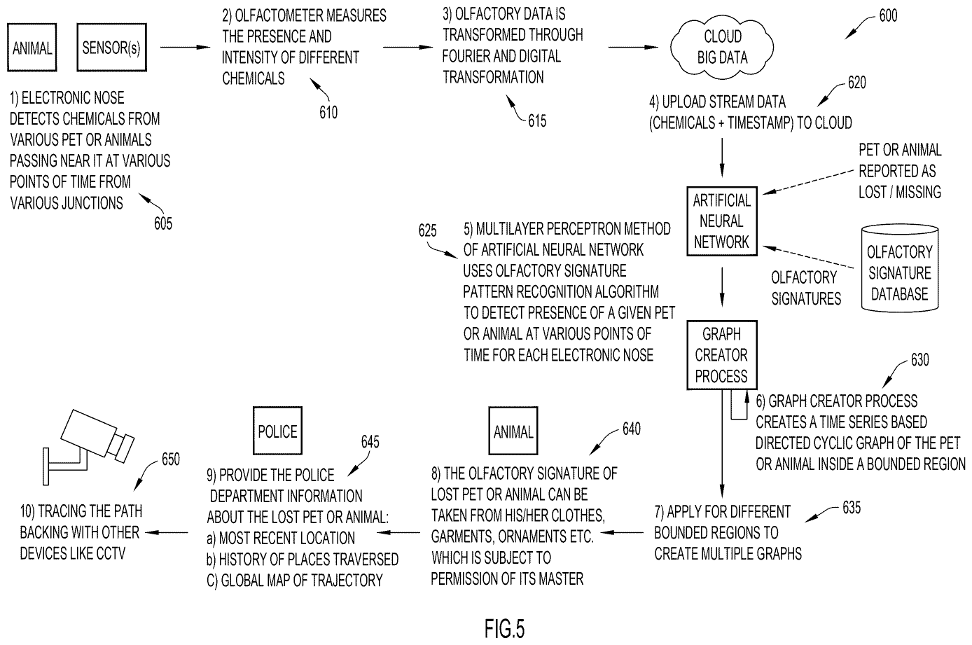

A flow diagram 600 of a manner of animal tracking (e.g., via animal tracking code 200, computer 101, etc.) according to an embodiment of the present invention is illustrated in FIG. 5. Initially, the chemical sensors are disposed at various locations in substantially the same manner described above. The chemical sensors detect chemicals from various pets or animals passing near them at various points of time from various junctions at flow 605. The chemical sensors (e.g., olfactometer, E-nose, etc.) measure the presence and intensity of different chemicals at flow 610 in substantially the same manner described above. The olfactory data is transformed through Fourier and digital transformation to produce the table data at flow 615 in substantially the same manner described above. The resulting stream of data of the chemicals and timestamp is stored (e.g., uploaded to the cloud) at operation 620 in substantially the same manner described above.

An olfactory signature of a lost pet or animal can be obtained from clothes, garments, ornaments, etc. associated with the lost pet or animal (e.g., which may be subject to the permission of its owner, etc.) at flow 640 in substantially the same manner described above. The machine learning model (e.g., artificial neural network, etc.) uses olfactory signature pattern recognition to detect the presence of a given pet or animal at various points of time for each chemical sensor at flow 625 in substantially the same manner described above.

A time series based directed cyclic graph of the pet or animal inside a bounded region is created at flow 630 in substantially the same manner described above. The graph creation is applied for different bounded regions to create multiple graphs at flow 635. Information about the lost pet or animal (e.g., most recent location, a history of places traversed, a global map of its trajectory, etc.) may be provided to an appropriate authority (e.g., police, etc.) at flow 645 to retrieve the lost pet or animal or perform other action. The path of the animal may be traced with other devices (e.g., CCTV, etc.) at flow 650. This may used to confirm the identity and/or location of the pet or animal in substantially the same manner described above.

Present invention embodiments provide various technical and other advantages. For example, an embodiment of the present invention provides non-invasive real time tracking of pets or animals. The sensor detections reduce computing time and resources by enabling fast identification of data in large datasets (e.g., CCTV or other time series data, etc.). Further, the system may be integrated with various sensors/systems (e.g., CCTV, GPS/GNSS, etc.) to improve accuracy with respect to location and timing.

In addition, the machine learning model may be continuously updated (or trained) based on feedback related to actual locations of the lost pet or animal. For example, a detection may initially be indicated with lower confidence or probability. The detection may be verified based on other devices (e.g., CCTV, GNSS/GPS, etc.). The verified detection may be used to update or train the machine learning model to increase the confidence for the detection (e.g., update or train the machine learning model to increase the probability of the detection, etc.). This updating or retraining of the machine learning model may also be performed for detections with high confidence or probability that are erroneous based on the verification. Thus, the machine learning model may continuously evolve (or be trained) to learn or improve detections (e.g., with varying conditions of the lost pet or animal altering the olfactory signature).

It will be appreciated that the embodiments described above and illustrated in the drawings represent only a few of the many ways of implementing embodiments for animal tracking based on chemical signature.

The environment of the present invention embodiments may include any number of computer or other processing systems (e.g., client or end-user systems, server systems, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system. These systems may include any types of monitors and input devices (e.g., keyboard, mouse, voice recognition, etc.) to enter and/or view information.

It is to be understood that the software of the present invention embodiments (e.g., animal tracking code 200, etc.) may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the server and/or client systems. The database systems and/or storage structures may be remote from or local to the computer or other processing systems, and may store any desired data.

The present invention embodiments may employ any number of any type of user interface (e.g., Graphical User Interface (GUI), command-line, prompt, etc.) for obtaining or providing information (e.g., olfactory signature or other characteristics of the pet or animal, graphs, trajectory, locations, times, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

A report may include any information arranged in any fashion, and may be configurable based on rules or other criteria to provide desired information to a user (e.g., olfactory signature or other characteristics of the pet or animal, graphs, trajectory, locations, times, etc.).

The present invention embodiments are not limited to the specific tasks or algorithms described above, but may be utilized for tracking any animate or inanimate objects with an identifiable chemical or other pattern.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
receiving, via at least one processor, information from sensors at different locations indicating detection of chemicals from a plurality of animals in proximity of the sensors;
determining, via a machine learning model of the at least one processor, presence of a pattern of chemicals associated with an animal within the information from the sensors; and
identifying, via the at least one processor, one or more locations of the animal based on the sensors detecting the pattern of chemicals associated with the animal.

2. The method of claim 1, wherein determining presence of a pattern of chemicals associated with the animal comprises:
converting the information from the sensors via a Fourier transform and comparing the pattern of chemicals associated with the animal to patterns within the converted information.

3. The method of claim 1, wherein the machine learning model includes a neural network.

4. The method of claim 1, further comprising:
generating, via the at least one processor, a graph indicating the one or more locations and paths traversed by the animal.

5. The method of claim 1, further comprising:
determining, via the at least one processor, a health condition of the animal based on the information from the sensors corresponding to the pattern of chemicals associated with the animal.

6. The method of claim 1, wherein the one or more locations are identified further based on one or more from a group of CCTV information and global positioning information.

7. The method of claim 1, wherein the chemical pattern associated with the animal is obtained from one or more objects used by the animal.

8. A computer system comprising:
a processor set;
one or more computer-readable storage media; and
program instructions stored on the one or more computer-readable storage media to cause the processor set to perform operations comprising:
receiving information from sensors at different locations indicating detection of chemicals from a plurality of animals in proximity of the sensors;
determining, via a machine learning model, presence of a pattern of chemicals associated with an animal within the information from the sensors; and
identifying one or more locations of the animal based on the sensors detecting the pattern of chemicals associated with the animal.

9. The computer system of claim 8, wherein determining presence of a pattern of chemicals associated with the animal comprises:
converting the information from the sensors via a Fourier transform and comparing the pattern of chemicals associated with the animal to patterns within the converted information.

10. The computer system of claim 8, wherein the machine learning model includes a neural network, and the chemical pattern associated with the animal is obtained from one or more objects used by the animal.

11. The computer system of claim 8, wherein the operations further comprise:
generating a graph indicating the one or more locations and paths traversed by the animal.

12. The computer system of claim 8, wherein the operations further comprise:
determining a health condition of the animal based on the information from the sensors corresponding to the pattern of chemicals associated with the animal.

13. The computer system of claim 14, wherein the one or more locations are identified further based on one or more from a group of CCTV information and global positioning information.

14. A computer program product comprising:
one or more computer-readable storage media; and
program instructions stored on the one or more computer-readable storage media to perform operations comprising:
receiving information from sensors at different locations indicating detection of chemicals from a plurality of animals in proximity of the sensors;
determining, via a machine learning model, presence of a pattern of chemicals associated with an animal within the information from the sensors; and
identifying one or more locations of the animal based on the sensors detecting the pattern of chemicals associated with the animal.

15. The computer program product of claim 14, wherein determining presence of a pattern of chemicals associated with the animal comprises:
converting the information from the sensors via a Fourier transform and comparing the pattern of chemicals associated with the animal to patterns within the converted information.

16. The computer program product of claim 14, wherein the machine learning model includes a neural network.

17. The computer program product of claim 14, wherein the operations further comprise:
generating a graph indicating the one or more locations and paths traversed by the animal.

18. The computer program product of claim 14, wherein the operations further comprise:
determining a health condition of the animal based on the information from the sensors corresponding to the pattern of chemicals associated with the animal.

19. The computer program product of claim 14, wherein the one or more locations are identified further based on one or more from a group of CCTV information and global positioning information.

20. The computer program product of claim 14, wherein the chemical pattern associated with the animal is obtained from one or more objects used by the animal.
